(19) United States
(12) Patent Application Publication
(10) Pub. No.: US 2022/0054510 A1
(43) Pub. Date: Feb. 24, 2022

(54) USE OF 1-ALPHA-HYDROXYVITAMIN D5 AND RELATED COMPOUNDS AS PROPHYLACTIC OR THERAPEUTIC AGENTS FOR SARS-COV-2 (COVID-19 VIRUS) AND RELATED INFECTIONS

(71) Applicants: Richard J. Pariza, Zion, IL (US); Robert M. Moriarty, Michiana Shores, IN (US)

(72) Inventors: Richard J. Pariza, Zion, IL (US); Robert M. Moriarty, Michiana Shores, IN (US)

(21) Appl. No.: 17/404,254

(22) Filed: Aug. 17, 2021

Related U.S. Application Data
(60) Provisional application No. 63/067,124, filed on Aug. 18, 2020.

(51) Int. Cl. A61K 31/592

(52) U.S. Cl. CPC A61K 31/592

(57) ABSTRACT

The use of 1-alpha-hydroxyvitamin D5 or related compounds as prophylactic or therapeutic agents for SARS-CoV-2 (Covid-19 virus) and related infections. The compounds may be administered orally, by inhalation, nasal spray, eye drops, or by injection. The compounds may be administered to persons who are unwilling, unable or adverse to be vaccinated. The compounds also may be administered to immunocompromised persons for whom vaccination may not provide adequate protection from infection.

### BACKGROUND OF THE INVENTION

### Field of the Invention

This disclosure relates to the use of 1-alpha-hydroxyvitamin D5 or related compounds as prophylactic or therapeutic agents for SARS-CoV-2 (Covid-19 virus) and related infections.

### Description of the Related Art

COVID-19, the disease caused by SARS-CoV-2, was declared a pandemic by the World Health Organization in March 2020. Some known antivirals are currently being used to manage the disease with limited success, as well as powerful new vaccines. Calcitriol has been shown to have in vitro activity against this virus (biorxiv.org/content/10.1101/2020.06.21.162396v1), but is well-known to be toxic in doses that may be necessary for effective treatment or prophylaxis. We herein describe compounds related to Calcitriol that are less toxic and therefore may be safely administered as therapeutic treatments for persons with Corona virus infections or as prophylactic agents to prevent infection by Corona viruses or as agents to limit the transmission of the infection by infected persons or animals. These agents may also be used by persons who are unwilling, unable or adverse to be vaccinated, or animals for which there may be no approved vaccine. These compounds may also be administered to immunocompromised persons for whom vaccination may not provide adequate protection from infection.

### BRIEF SUMMARY OF THE INVENTION

The present disclosure relates to the use of 1-alpha-hydroxyvitamin D5 (CAS#187935-17-7; U.S. Pat. No. 6,900,191 (2005), and Journal of Organic Chemistry, 2005, 70(19), 7624) and related compounds as safe and effective treatments for patients who are Covid-19 positive, who have or do not have obvious symptoms, as well as preventative prophylactics to be taken by Covid-19 negative persons, or persons of unknown status, who may come into contact with the virus. FIG. 2 shows that 1-alphahydroxyvitamin D5 completely (greater than 99%) eliminated the SARS-CoV-2 virus infection in Ace2-A549 cells (human lung epithelial cells) at a concentration of 10 micromolar, without killing the host cells.

These compounds may preferentially be administered orally, by inhalation, nasal spray, eye drops, or by injection, and may be taken by persons who are unwilling, unable or adverse to be vaccinated, or animals for which there may be no approved vaccine. These compounds may also be administered to immunocompromised persons for whom vaccination may not provide adequate protection from infection. New strains, such as the delta strain, can harbor in the nasal passages of vaccinated persons, as well as unvaccinated persons, and therefore be transmitted to others. These compounds may be administered to reduce the viral load in the airways of infected persons or animals to decrease or eliminate the risk of transmission to others.

### BRIEF DESCRIPTION OF THE DRAWINGS

FIG. 1 illustrates the chemical structure of 1-alpha-hydroxyvitamin D5 or related compounds of interest.

FIG. 2 is a diagram illustrating in vitro results of treatment of Ace2-A549 cells (human lung epithelial cells) infected by SARS-CoV-2 with 1-alpha-hydroxyvitamin D5 at The University of Chicago, Department of Microbiology, Chicago, Ill.

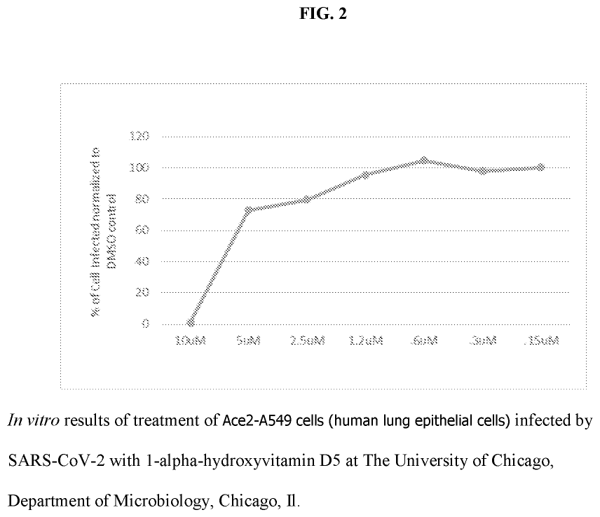

### DETAILED DESCRIPTION OF THE INVENTION

While the invention described herein may be embodied in many forms, there is shown in the figures and will herein be described in detail one or more embodiments with the understanding that this disclosure is to be considered an exemplification of the principles of the invention and is not intended to limit the disclosure to the illustrated embodiments. Aspects of the different embodiments can be combined with or substituted for one another.

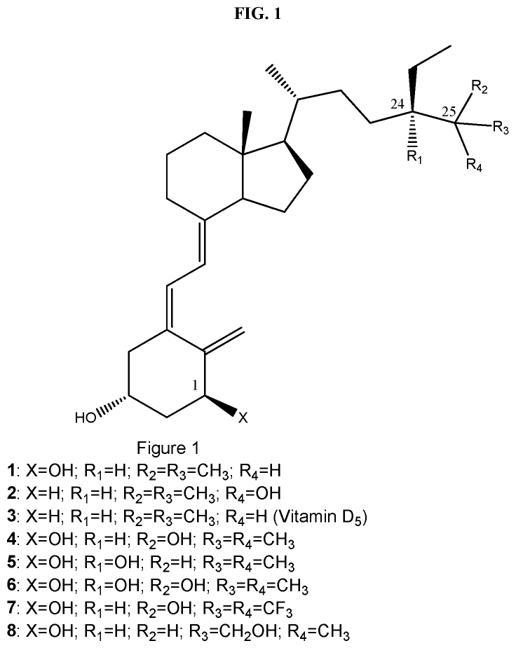

We have developed the use of 1-alpha-hydroxyvitamin D5 or related compounds, (for examples, see FIG. 1), as safe and effective therapeutic treatments for patients who are Covid-19 positive, who have or do not have obvious symptoms, as well as preventative prophylactics to be taken by Covid-19 negative persons, or persons of unknown status, who may come into contact with the virus. These compounds may preferentially be administered orally, by inhalation, nasal spray, eye drops, or by injection, and may be taken by persons who are unwilling, unable or adverse to be vaccinated, or animals for which there may be no approved vaccine. These compounds may also be administered to immunocompromised persons for whom vaccination may not provide adequate protection from infection. New strains, such as the delta strain, can harbor in the nasal passages of vaccinated persons, as well as unvaccinated persons, and therefore be transmitted to others. These compounds may be administered to reduce the viral load in the airways of infected persons or animals to decrease or eliminate the risk of transmission to others.

Thus, a method for treating a patient who is Covid-19 positive is provided. The method comprises administering to the patient a pharmaceutically effective amount of 1-alpha-hydroxyvitamin D5 or related compounds. The 1-alpha-hydroxyvitamin D5 or related compounds may be administered orally, by inhalation, nasal spray, eye drops, or by injection. The 1-alpha-hydroxyvitamin D5 or related compounds may be administered to persons who are unwilling, unable or adverse to be vaccinated. The 1-alpha-hydroxyvitamin D5 or related compounds may be administered to immunocompromised persons for whom vaccination may not provide adequate protection from infection. The 1-alpha-hydroxyvitamin D5 or related compounds may be administered to reduce the viral load in the airways of infected persons or animals to decrease or eliminate the risk of transmission to other persons or animals. The 1-alpha-hydroxyvitamin D5 or related compounds may be administered to patients who have an infection from a Corona virus or who have or do not have obvious symptoms.

In another aspect a method for preventing Covid-19 in a Covid-19 negative person or a person of unknown Covis-19 status is provided. The method comprises administering to the person a pharmaceutically effective amount of 1-alpha-hydroxyvitamin D5 or related compounds as a preventative prophylactic. The 1-alpha-hydroxyvitamin D5 or related compounds may be administered orally, by inhalation, nasal spray, eye drops, or by injection. The 1-alpha-hydroxyvitamin D5 or related compounds may be administered to persons who are unwilling, unable or adverse to be vaccinated. The 1-alpha-hydroxyvitamin D5 may be administered to immunocompromised persons for whom vaccination may not provide adequate protection from infection. The 1-alpha-hydroxyvitamin D5 may be administered to reduce the viral load in the airways of infected persons or animals to decrease or eliminate the risk of transmission to other persons or animals.

It is understood that the embodiments of the invention described above are only particular examples which serve to illustrate the principles of the invention. Modifications and alternative embodiments of the invention are contemplated which do not depart from the scope of the invention as defined by the foregoing teachings and appended claims. It is intended that the claims cover all such modifications and alternative embodiments that fall within their scope.

1. A method for treating a patient who is Covid-19 positive, the method comprising administering to the patient a pharmaceutically effective amount of 1-alpha-hydroxyvitamin D5 or related compounds.

2. The method of claim 1 wherein the 1-alpha-hydroxyvitamin D5 or related compounds are administered orally, by inhalation, nasal spray, eye drops, or by injection.

3. The method of claim 1 wherein the 1-alpha-hydroxyvitamin D5 or related compounds is administered to persons who are unwilling, unable or adverse to be vaccinated.

4. The method of claim 1 wherein the 1-alpha-hydroxyvitamin D5 or related compounds is administered to immunocompromised persons for whom vaccination may not provide adequate protection from infection.

5. The method of claim 1 wherein the 1-alpha-hydroxyvitamin D5 or related compounds is administered to reduce the viral load in the airways of infected persons or animals to decrease or eliminate the risk of transmission to other persons or animals.

6. The method of claim 1 wherein the 1-alpha-hydroxyvitamin D5 or related compounds is administered to patients who have an infection from a Corona virus or who have or do not have obvious symptoms.

7. A method for preventing Covid-19 in a Covid-19 negative person or a person of unknown Covis-19 status, the method comprising administering to the person a pharmaceutically effective amount of 1-alpha-hydroxyvitamin D5 or related compounds as a preventative prophylactic.

8. The method of claim 7 wherein the 1-alpha-hydroxyvitamin D5 or related compounds is administered orally, by inhalation, nasal spray, eye drops, or by injection.

9. The method of claim 7 wherein the 1-alpha-hydroxyvitamin D5 or related compounds is administered to persons who are unwilling, unable or adverse to be vaccinated.

10. The method of claim 7 wherein the 1-alpha-hydroxyvitamin D5 is administered to immunocompromised persons for whom vaccination may not provide adequate protection from infection.

11. The method of claim 7 wherein the 1-alpha-hydroxyvitamin D5 is administered to reduce the viral load in the airways of infected persons or animals to decrease or eliminate the risk of transmission to other persons or animals.
